(19) United States
(12) Patent Application Publication
(10) Pub. No.: US 2009/0048121 A1
(43) Pub. Date: Feb. 19, 2009

(54) Screening Method for the Early Diagnosis of Cerebral Vasospasm

(75) Inventors: Martin Maurer, Heidelberg (DE); Wolfgang Kuschinsky, Heidelberg (DE); Daniel Haux, Heidelberg (DE); Oliver Sakowitz, Ladenburg (DE); Robert Feldmann, Wiesbaden (DE); Andreas Unterberg, Heidelberg (DE)

Correspondence Address: SUZANNAH K. SUNDBY;SMITH, GAMBRELL & RUSSEL, LLP, 1130 Connecticut Avenue, NW, Suite 1130, WASHINGTON, DC, 20036, US

(73) Assignee: RUPRECHT-KARLS-UNIVERSITAT HEIDELBERG, Heidelberg (DE)

(21) Appl. No.: 11/718,103

(22) PCT Filed: Oct. 31, 2005
(86) PCT No.: PCT/EP2005/011804

§ 371 Date: Apr. 27, 2007

(30) Foreign Application Priority Data
Nov. 2, 2004 (DE) 10 2004 052 889.6

(51) Int. Cl. C40B 30/06; C12Q 1/32; G01N 33/573; C12M 1/00

(52) U.S. Cl. 506/10; 435/26; 435/7.4; 435/287.2

(57) ABSTRACT

The invention relates to screening methods and test systems for the in vitro diagnosis and detection of an increased risk of vasospasm in a patient. In addition, the invention describes the use of GAPDH and/or Hsc70 to diagnose an increased risk of vasospasm. Also described is a method for identifying a pharmaceutically active substance for the prevention and/or treatment of cerebral vasospasm and a method for screening a substance library to identify a pharmaceutically active substance for the prevention and/or treatment of cerebral vasospasm. Pharmaceutically active substances and combination preparations are also disclosed.

### FIELD OF THE INVENTION

The present invention relates to the technical field of screening methods and test systems. In particular, the invention relates to a method for the in vitro diagnosis and to a test system for detecting an increased risk of vasospasm in a patient. In addition, the invention describes the use of glyceraldehyde-3-phosphate dehydrogenase protein (GAPDH) and/or heat shock cognate 70 kDa protein (Hsc70) to diagnose an increased risk of vasospasm. Also described is a method for identifying a pharmaceutically active substance for the prevention and/or treatment of cerebral vasospasm and a method for screening a substance library to identify a pharmaceutically active substance for the prevention and/or treatment of cerebral vasospasm. Pharmaceutically active substances and combination preparations are also disclosed.

### BACKGROUND OF THE INVENTION

Stroke is one of the most frequent causes of death and disability. Around 85% of stroke patients suffer from vascular occlusions, known as ischemic strokes, while in 15% of patients a brain hemorrhage is the cause of their illness. Besides hemorrhaging in the brain parenchyma, most cases are caused by a hemorrhage in the subarachnoid space, known as a subarachnoid hemorrhage (SAH). SAH contributes to 6-8% of all strokes and is responsible for 22-25% of all cerebrovascular deaths (Dumont et al., Neurosurgery 53 (2003), 123-133; Discussion 33-35).

The causes of SAH are 60% intracranial aneurysms, 10% arteriovenous malformations and 10% rarer causes such as e.g. hemorrhagic diathesis, anticoagulant therapy, tumors or vasculitides. Around 20% of cases remain etiologically unexplained. It is estimated that 1-12 million people in the United States of America carry a cerebral aneurysm, with an annual prevalence of SAH of 30,000 people (Dumont et al., Neurosurgery 53 (2003), 123-133).

Each year, around 7-20 per 100,000 inhabitants suffer an SAH (data for North America and Europe) (Dumont et al., Neurosurgery 53 (2003), 123-133). SAH has a high mortality rate—around 15% of patients die even before reaching hospital, a further 15% die within the first 24 hours, a further 15% die within the first 2 weeks and a further 15% die within the next 2 months. After 2 years, only 15% of SAH patients are still alive, most of these having their daily life affected by a high level of disability.

Apart from restricting the life of the patient, the costs incurred during the stay in hospital are very high due to the long period of treatment in the intensive care unit. Added to this is the financial damage caused by loss of earnings and pension.

Apart from post-operative hemorrhaging and occlusion hydrocephalus, the main complications of SAH consist primarily in secondary ischemia, which is caused by symptomatic cerebral vasospasm. Synonymously with this, mention is made of delayed ischemic neurological deficit syndrome (DINDS) (Hijdra et al., Stroke 18 (1987), 1061-1067). An angiographically detected narrowing of the vessels occurs as a complication in around 70% of cases, and clinically manifest vasospasm occurs in about 20-30% of cases (Dumont et al., Neurosurgery 53 (2003), 123-133; Discussion 33-35; Janjua and Mayer, Curr Opin Crit Care 9 (2003), 113-119). Women are affected by this much more frequently than men. Despite maximum therapy, around 50% of vasospasm patients develop an additional ischemic brain infarct. The maximum symptomatic vasospasm occurs around 7-14 days after the SAH, so that there is time for treatment/prophylaxis between hemorrhaging and vasospasm.

The pathogenesis of cerebral vasospasm is complicated. Various models and influencing factors are being discussed, including (1) free hemoglobin in the subarachnoid space; (2) inflammation reactions to the hemorrhage, mediated by cytokines such as TNF-α, IL-1α, IL-11, IL-6 and IL-8, immunoglobulins, NFkB, leucocytes and leucocyte adhesion molecules, COX-2, ET-1 and PARP; (3) an abnormal NO metabolism; (4) changes to the activity of protein kinase C; (5) the formation of free radicals; (6) the release of prostaglandins; (7) the degradation of filament-associated proteins (Dietrich and Dacey, Neurosurgery 46 (2000), 517-530; Dumont et al., J Neurosurgery 96 (2002), 985-986; Discussion 6-7; Dumont et al., Neurosurgery 53 (2003), 123-133; Janjua and Mayer, Curr Opin Crit Care 9 (2003), 113-119; Laher and Zhang, J Cereb Blood Flow Metab 21 (2001), 887-906; Sobey, Clin Exp Pharmacol Physiol 28 (2001), 926-929).

EP 1 122 317 A1 describes a monoclonal antibody against GAPDH, a hybridoma for preparing it and the use of this antibody for the treatment of various neurodegenerative diseases such as, for example, Alzheimer's disease, Parkinson's disease and Creutzfeldt-Jakob disease.

WO 01/52890 A1 describes a complex consisting of a heat shock protein and an antigenic molecule which shows the antigenicity of an antigen associated with a neurodegenerative disorder. The heat shock protein may be the Hsp70 protein. As neurodegenerative disorders, mention may be made inter alia of Alzheimer's disease, Parkinson's disease, Creutzfeldt-Jakob disease, stroke and brain trauma.

It is presently not known why vasospasm occurs in some patients after an SAH but not in others. Since maximum vasospasm does not occur until around 7-14 days after SAH, there is time beforehand to prevent vasospasm by means of treatment/prophylaxis. However, a prerequisite for this is to find the patients which are highly likely to suffer from vasospasm.

There is no early indicator for vasospasm. At present, vasospasm can be detected only after it has occurred, by means of changes in the clinical picture, imaging methods (CT, MRT), blood flow tests (Doppler sonography) and by changes to metabolic parameters in cerebral microdialysis (pyruvate, lactate, glutamate) (Berger et al., Nervenarzt 75 (2004), 113-123).

All the methods known to date permit diagnosis only after vasospasm has occurred, so that therapeutic intervention in good time is not possible.

There is therefore a need for methods for the in vitro diagnosis and test systems for the detection of an increased risk of vasospasm. The object of the present invention is therefore to provide a method which allows early diagnosis of cerebral vasospasm.

Using molecular indicators, the present invention makes it possible to detect the patient's risk after SAH even before vasospasm occurs, to monitor the patient closely and to initiate prophylaxis in good time.

### SUMMARY OF THE INVENTION

The present invention relates to a method for the in vitro diagnosis of an increased risk of vasospasm in a patient, comprising the steps:
(a) providing a sample from the patient;
(b) determining the quantity of glyceraldehyde-3-phosphate dehydrogenase protein (GAPDH) and/or heat shock cognate 70 kDa protein (Hsc70) in the test sample;
(c) comparing the quantity of GAPDH and/or Hsc70 with a reference value;
   wherein a GAPDH quantity that is higher than the reference value and/or an Hsc70 quantity that is lower than the reference value indicates an increased risk of vasospasm.

The present invention further relates to a test system for detecting an increased risk of vasospasm after a stroke, comprising:
(a) at least one antibody against GAPDH and/or Hsc70;
(b) at least one secondary antibody for detecting antibody/GAPDH and/or antibody/Hsc70 complexes;
(c) optionally means for quantifying the complexes;
   wherein a quantity of GAPDH that is higher than the reference value and/or a quantity of Hsc70 that is lower than the reference value indicates an increased risk of vasospasm.

The invention also relates to a method for identifying a pharmaceutically active substance for the prevention and/or treatment of cerebral vasospasm, comprising the steps:
(a) providing a cell which expresses GAPDH and/or Hsc70;
(b) bringing a test substance into contact with the cell;
(c) measuring the quantity of GAPDH and/or Hsc70;
   wherein a reduction in the quantity of GAPDH and/or an increase in the quantity of Hsc70 qualifies the test substance as a pharmaceutically active substance.

Also encompassed is a method for screening a substance library to identify a pharmaceutically active substance for the prevention and/or treatment of cerebral vasospasm, comprising the steps:
(a) providing test samples containing cells which express GAPDH and/or Hsc70;
(b) measuring the initial expression quantity of GAPDH and/or Hsc70;
(c) bringing the test samples into contact with candidates from the substance library;
(d) identifying a test sample which expresses less GAPDH and/or more Hsc70 compared to the initial expression quantity;
(e) determining the candidate from the substance library which was brought into contact with the test sample,
   wherein the candidate from the substance library is a pharmaceutically active substance.

In addition, the invention relates to a pharmaceutically active substance obtainable by one of the aforementioned methods, and to a combination preparation comprising an inhibitor of GAPDH and an activator of Hsc70 for the prevention and/or prophylaxis of cerebral vasospasm.

### BRIEF DESCRIPTION OF THE FIGURES

FIG. 1 shows two-dimensional electropherograms of human microdialysate for the asymptomatic SAH patients (left-hand half) and for the patients with vasospasm (right-hand half). Differentially expressed protein groups were identified by hierarchical cluster analysis and are marked (arrows) for (A) heat shock cognate 70 kDa protein (Hsc70) and (B) glyceraldehyde-3-phosphate dehydrogenase (GAPDH).

FIG. 2 shows a hierarchical cluster analysis of the proteome profiles. The cluster algorithm sorts the protein spots according to their expression profile. The experiment numbers are located at the top (n=24) and the spot numbers are located to the right. Differential clusters are marked for Hsc70 and GAPDH.

FIG. 3 shows an increased expression of GAPDH in the microdialysate of vasospastic patients after SAH. The average protein concentration of GAPDH was increased by a factor of 3.68±1.09 in the patients of the vasospasm group compared to the asymptomatic control patients.

FIG. 4 shows a reduced expression of Hsc70 in the microdialysate of vasospastic patients after SAH. The average protein concentration of Hsc70 was reduced by a factor of 0.47±0.18 in the patients of the vasospasm group compared to the asymptomatic control patients.

FIG. 5 shows changes in the microdialysate prior to the occurrence of vasospasm. On average, protein changes in the microdialysate were found 2.4±2.1 days before the clinical occurrence of vasospasm.

### DETAILED DESCRIPTION OF THE INVENTION

The present invention firstly relates to methods for the in vitro diagnosis of an increased risk of vasospasm in a patient, comprising the steps:
(a) providing a sample from the patient;
(b) determining the quantity of glyceraldehyde-3-phosphate dehydrogenase protein (GAPDH) and/or heat shock cognate 70 kDa protein (Hsc70) in the test sample;
(c) comparing the quantity of GAPDH and/or Hsc70 with a reference value;
   wherein a GAPDH quantity that is higher than the reference value and/or an Hsc70 quantity that is lower than the reference value indicates an increased risk of vasospasm.

By means of proteome analysis based on two-dimensional gel electrophoresis and mass spectrometry, the present invention has identified two new marker proteins for cerebral vasospasm in the microdialysate. These are glyceraldehyde-3-phosphate dehydrogenase (GAPDH) and the heat shock cognate 70 kDa protein (Hsc70). The GAPDH concentration was 3.68±1.09 (data from 4 protein isoforms), i.e. more than three times, higher in the patients of the vasospasm group compared to the asymptomatic control patients, while the Hsc70 concentration was 0.47±0.18 (data from 9 protein isoforms), i.e. around a half, lower.

The two aforementioned proteins were not previously associated with cerebral vasospasm and especially the early diagnosis thereof.

The significant advantages of the present invention consist in that the risk of developing vasospasm after SAH can be assessed prior to any worsening of the clinical condition, and early prophylaxis and/or treatment steps can be initiated accordingly.

Besides the known enzymatic functions of GAPDH in glucose metabolism, in recent years the interest in this protein has increasingly focused on its role in cell death and apoptosis, interaction with NO, endocytosis, microtubule bundling, phosphotransferase activity, regulation of gene expression, transport of intranuclear RNA, DNA replication and repair and also translational regulation processes (Sirover, J Cell Biochem 66 (1997), 133-140; Sirover, Biochim Biophys Acta 1432 (1999), 159-184).

Reduced GAPDH activity is also found in many neurodegenerative diseases such as, for example, Alzheimer's disease, Huntington's disease, dentatorubral pallidoluysian atrophy and other CAG triplet disorders. GAPDH is also the target molecule of deprenyl, a substance which is used for the treatment of Parkinson's disease and which has a neuroprotective effect (Kragten et al., J Biol Chem 273 (1998), 5821-5828; Tatton et al., J Neural Transm 110, (2003), 509-515).

Nuclear translocation presumably leads to the apoptotic signals which are responsible for the cell death of neurons (Chuang and Ishitani, Nat Med 2 (1996), 609-610; Sawa et al., Proc Natl Acad Sci USA 94 (1997), 11669-11674; Sunaga et al., Neurosci Lett 200 (1995), 133-136; Tatton et al., J Neural Transm Suppl (2000), 77-100). Furthermore, the expression of GAPDH can be regulated by hypoxia (Graven et al., Am J Physiol 274 (1998), C347-55; Graven et al., J Biol Chem 269 (1994), 24446-24453). GAPDH forms protein complexes with TOAD64, enolase-χ, aldolase C and Hsc70 (Bulliard et al., Biochem J 324 (Pt 2) (1997), 555-563), and these protein complexes play a role in membrane fusion and intracellular signal pathways. GAPDH can also be stimulated by oxyhemoglobin (Brookes et al., FEBS Lett 416 (1997), 90-92).

Hsc70 belongs to the group of molecular chaperones of the Hsp70 family. This protein family is found in the cell nucleus and cytosol and serves as a folding aid for growing polypeptide chains. They also prevent an accumulation of incorrectly folded proteins. Hsc70 is found both in neurons and in glial cells in the brain (Myung et al., Mol Genet Metab 80 (2003), 444-450) and forms complexes with heat shock protein 90 kDa (Hsp90), glyceraldehyde-3-phosphate dehydrogenase (GAPDH), leucinaminopeptidase (LAP) and adenosylhomocysteinase (Nakamura et al., Biochem Biophys Res Commun 290 (2002), 858-864). Oxidative stress caused by hypoxic-ischemic conditions leads to nuclear translocation of Hsc70 (Dastoor and Dreyer, J Cell Sci 113 (Pt 16) (2000), 2845-2854) and of GAPDH (Sawa et al., Proc Natl Acad Sci USA 94 (1997), 11669-11674), which both promote apoptotic processes in the affected cells.

Without being restricted by any theory, the inventors of the present invention assume that, in addition to their ability to bind to one another, there is also a functional relationship between the two proteins GAPDH and Hsc70. This relationship could be provided via the NO metabolism. Both proteins react with NO, and there are less reaction products of NO (NOₓ) in the microdialysate of patients with vasospasm after SAH compared to non-vasospastic control patients (Sakowitz et al., J Cereb Blood Flow Metab 21 (2001), 1067-1076).

In one preferred embodiment of the invention, the sample from the patient is a cerebral microdialysate, cerebrospinal fluid, blood plasma or blood serum.

The microdialysate can be obtained by microdialysis. Microdialysis is a method used to collect the components of the extracellular fluid in living tissue (Benveniste, J Neurochem 52 (1989), 1667-1679; Ungerstedt, Measurement of neurotransmitter release in vivo. 6, Marsden C A ed. Chishester, Wiley (1984), 81-105; Ungerstedt, J Int Med 230 (1991), 365-373). It has been used in stroke patients mainly to monitor the progress of neurotransmitters and small metabolites of the brain (Berger et al., Nervenarzt 75 (2004), 113-123).

Several studies have previously used cerebral microdialysis to examine progress after SAH (Peerdeman et al., J Neurol 250 (2003), 797-805). As markers of an abnormal cerebral metabolism due to symptomatic vasospasm, use has been made in particular of glutamate, lactate and the lactate/pyruvate quotient (Persson and Hillered, J Neurosurg 76 (1992), 72-80; Saveland et al., Neurosurgery 38 (1996), 12-19; Sarrafzadeh et al., Crit Care Med 30 (2002), 1062-1070; Unterberg et al., J Neurosurg 94 (2001), 740-749; Sarrafzadeh et al., Stroke 35 (2004), 638-643).

However, cerebral microdialysis has until now been limited to specialized centers with the necessary technical prerequisites, for which reason the number of patients remains limited (Janjua and Mayer, Curr Opin Crit Care 9 (2003), 113-119). Furthermore, in the event of an incorrectly placed measurement catheter, microdialysis as a local measurement method is often unable to provide an early indication of an ischemic metabolic disorder due to vasospasm (Sarrafzadeh et al., Curr Neurol Neurosci Rep 3 (2003), 517-523).

In a further preferred embodiment, the increased risk of vasospasm occurs after a subarachnoid hemorrhage (SAH) or after an intracerebral hemorrhage.

In a further embodiment, the determination of the quantity of GAPDH and/or Hsc70 takes place by means of a protein concentration determination.

The protein concentration determination may advantageously take place by means of an antibody or by means of two-dimensional gel electrophoresis. Both monoclonal and polyclonal antibodies having a suitable specificity for GAPDH and/or Hsc70 can be used. Monoclonal antibodies are preferred on account of their higher specificity and simple preparation. Synthetically produced antibodies can also be used.

Two-dimensional gel electrophoresis is a method used to simultaneously examine an extremely large number of proteins in a sample (Gorg et al., Electrophoresis 21 (2000), 1037-1053; Rabilloud, Proteomics 2 (2002), 3-10). It is one of the most important methods in proteome research. The term “proteome” refers to the totality of all the proteins in a biological sample, a cell, an organ or an organism.

Protein determination by means of two-dimensional gel electrophoresis is particularly advantageous in this connection, wherein use may be made in particular of a method, which can be used to display and examine the proteins contained in the microdialysate, by means of a proteome study.

This method is based on two-dimensional gel electrophoresis in combination with mass spectrometry, whereby the inventors of the present invention have set up a reference database and a reference protein map for human cerebral microdialysate (Maurer et al., Proteome Sci 1 (2003), 7).

In a further embodiment of the invention, the vasospasm is cerebral vasospasm.

A further object of the present invention is the use of GAPDH and/or Hsc70 for diagnosing an increased risk of vasospasm. The vasospasm is preferably cerebral vasospasm.

GAPDH and/or Hsc70 can preferably be used to prepare a drug or a medicinal product for the prevention and/or treatment of cerebral vasospasm.

A further object of the present invention is a test system for detecting an increased risk of vasospasm after a stroke, comprising:
(a) at least one antibody against GAPDH and/or Hsc70;
(b) at least one secondary antibody for detecting antibody/GAPDH and/or antibody/Hsc70 complexes;
(c) optionally means for quantifying the complexes;
   wherein a quantity of GAPDH that is higher than the reference value and/or a quantity of Hsc70 that is lower than the reference value indicates an increased risk of vasospasm.

A further object of the present invention is a method for identifying a pharmaceutically active substance for the prevention and/or treatment of cerebral vasospasm, comprising the steps:
(a) providing a cell which expresses GAPDH and/or Hsc70;
(b) bringing a test substance into contact with the cell;
(c) measuring the quantity of GAPDH and/or Hsc70;
   wherein a reduction in the quantity of GAPDH and/or an increase in the quantity of Hsc70 qualifies the test substance as a pharmaceutically active substance.

With particular preference, the pharmaceutically active substance may be a drug or a medicinal product.

A further object of the present invention relates to a method for screening a substance library to identify a pharmaceutically active substance for the prevention and/or treatment of cerebral vasospasm, comprising the steps:
(a) providing test samples containing cells which express GAPDH and/or Hsc70;
(b) measuring the initial expression quantity of GAPDH and/or Hsc70;
(c) bringing the test samples into contact with candidates from the substance library;
(d) identifying a test sample which expresses less GAPDH and/or more Hsc70 compared to the initial expression quantity;
(e) determining the candidate from the substance library which was brought into contact with the test sample,
   wherein the candidate from the substance library is a pharmaceutically active substance.

In a preferred embodiment, high-throughput screening (HTS) is used for this method.

In a further preferred embodiment, the method comprises colorimetric, luminometric, fluorescence-based or radioactive methods.

A further object of the invention is a pharmaceutically active substance obtainable by one of the described methods.

It is unsolved why symptomatic vasospasm occurs in some patients and not in others. For this reason, it is at present recommended to treat all patients with the calcium antagonist nimodipin. Originally this was assumed to have a vessel-relaxing effect, but nowadays it is assumed to have a direct neuroprotective effect. Administration of nimodipin may on the one hand lead to unnecessary treatment of patients who will not develop vasospasm and on the other hand the fact that it lowers the blood pressure may promote vasospasm, so that the existing therapy cannot be considered satisfactory. In principle, only the so-called triple-H therapy is available at present for treating symptomatic vasospasm. This therapy consists of induced hypertension, hemodilution and hypervolemia by means of catecholamines and plasma expanders, but this is not recommended as prophylaxis due to the possible side effects. It is therefore possible to provide treatment only once neurological symptoms of vasospasm have appeared. Particularly in patients with relatively severe SAH who can be clinically/neurologically assessed only to a limited extent, valuable time is thus lost. The early dilation of the spastic vessels using a balloon catheter, which is presently being tested in the USA, is in the experimental stage. By virtue of the present invention it has become possible to start maximum therapy on selected patients earlier than before.

In one particularly preferred embodiment, the pharmaceutically active substance comprises an inhibitor of GAPDH, in particular an inhibitor which affects a metabolism pathway in which GAPDH is involved.

In a further preferred embodiment, the pharmaceutically active substance comprises an activator of Hsc70, in particular an activator which affects a metabolism pathway in which Hsc70 is involved.

A further object of the present invention is a combination preparation comprising an inhibitor of GAPDH and an activator of Hsc70 for the prevention and/or prophylaxis of cerebral vasospasm. The combination preparation has the particular advantage of increased effectiveness together with a reduced risk for the patient in the prevention and/or prophylaxis of cerebral vasospasm. By means of the combination preparation, individual doses of the inhibitor of GAPDH and of the activator of Hsc70 can be reduced in comparison with the individual preparations. This effect can probably be attributed to a combination effect of the two substances.

Examples which serve to explain the invention are given below. These examples are not intended to limit the scope of protection of the invention.

### Material and Methods Used

### 1. Human Cerebral Microdialysis

After obtaining approval for the study from the competent ethics commission and the consent of the patient or of the latter's appointed relatives, human cerebral microdialysate was obtained by the standard method known to the person skilled in the art (Sarrafzadeh et al., Crit Care Med, 30(5) (2002), 1062-1070). A flexible microdialysis catheter with a formal molecular weight barrier of 20 kDa (membrane diameter 0.65 mm, membrane length 10 mm) (CMA 70 custom probes, CMA Microdialysis, Solna, Sweden) was implanted in the fronto-parietal parenchyma of 97 patients who had all suffered a subarachnoid hemorrhage (SAH). After operative elimination of the aneurysm, the probes were put in place within 72 hours after the time of hemorrhage.

18 of the patients went on to develop symptomatic vasospasm (synonym: delayed neurological deficit syndrome (DINDS)). The concentrations of glutamate, lactate and the lactate/pyruvate quotient differ significantly from those of the patients who did not show any sign of vasospasm.

The microdialysis catheters were perfused with a sterile Ringer's solution at a flow rate of 0.3 μl/min and a sample was taken every 2 hours.

### 2. Proteome Analysis

The protein composition of the samples was tested by means of two-dimensional gel electrophoresis (2DE). In the first dimension, all the proteins of the sample are separated according to their isoelectric point, and in the second dimension they are separated according to their molecular weight. The 2DE was carried out according to the standard method known to the person skilled in the art (Berger et al., Nervenarzt 75 (2004), 113-123; Maurer et al., Proteome Sci 1 (2003), 7). To this end, the microdialysis samples were centrifuged at maximum speed for 5 min in a table-top centrifuge, in order to eliminate any disruptive substances. 5 to 10 μl (corresponding to approximately 1-2 μg of protein) of the sample were suspended in 6 mol/l urea, 2 M thiourea, 2% 3-[(3-cholamidopropyl)dimethyl-ammonio]-1-propane sulfonate (CHAPS), 0.5% IPG buffer pH 3-10 (Amersham Biosciences, Uppsala, Sweden) and a few grains of bromophenol blue in a total volume of 350 μl. The samples were applied to gel strips with an immobilized non-linear pH gradient from pH 3 to pH 10 (Immobiline DryStrip pH 3-10 NL, 18 cm) and isoelectrically focused in the IPGphor device (Amersham Biosciences, Uppsala, Sweden). The protocol for the isoelectric focusing consisted of 12 h swelling time at 30 V, then 200 V, 500 V and 1000 V for 1 h each. The voltage was then increased to 8000 V over 30 min and kept constant at 8000 V for a further 12 h, resulting in a total of 100300 Vh.

The gel strips were then incubated for 20 min in each of 1% dithiothreitol and 2.5% iodacetamide and transferred to a 12.5% polyacrylamide gel electrophoresis in the presence of 0.1% sodium dodecyl sulfate. Electrophoresis was carried out on the 20×20 cm² gels at 30 mA for 30 min and 100 mA for approximately 4 h in a suitable gel chamber. The gels were developed by highly sensitive silver staining in order to make the protein spot visible (Blum et al., Electrophoresis 8 (1987), 93-99), digitized and evaluated by densitometry using the Phoretix 2D Elite Software, Version 6.01 (Nonlinear Dynamics, Newcastle-upon-Tyne, UK). Individual protein spots were detected in the gels and their spot volume was determined, said spot volume being defined as the integral of the optical density over the spot surface area.

For each patient, 2 gel samples which had been taken at different times were tested.

### 3. Hierarchical Cluster Analysis

The raw data concerning the spot volumes were examined using the subprogram EPCLUST Version 0.9.23 beta of the EMBL-EBI program “Expression Profiler”, which can be called up from the Internet at the URL http://ep.ebi.ac.uk (Vilo et al., The analysis of gene expression data: Methods and software (2003)). The data were log 2-transformed and centered on the mean for each spot. All the data from the 20 gels (2 samples from each of 10 patients) were included in the analysis. The parameters of the hierarchical clustering were set to “average linkage (average distance, UPGMA)”, and linear correlation based on the linear correlation distance centered according to Pearson was used. The dendrogram cutting height was selected as 0.5.

### Results

On average, 57±22 protein spots (N=20) were found in the individual gels with a minimum of 37 up to a maximum of 149 protein spots. Hierarchical cluster analysis of the data showed 2 groups of differentially expressed proteins, which were identified by comparison with the proteome reference map for human microdialysate (Maurer et al., Proteome Sci 1 (2003), 7) as glyceraldehyde-3-phosphate dehydrogenase (GAPDH) and the heat shock cognate 70 kDa protein (Hsc70).

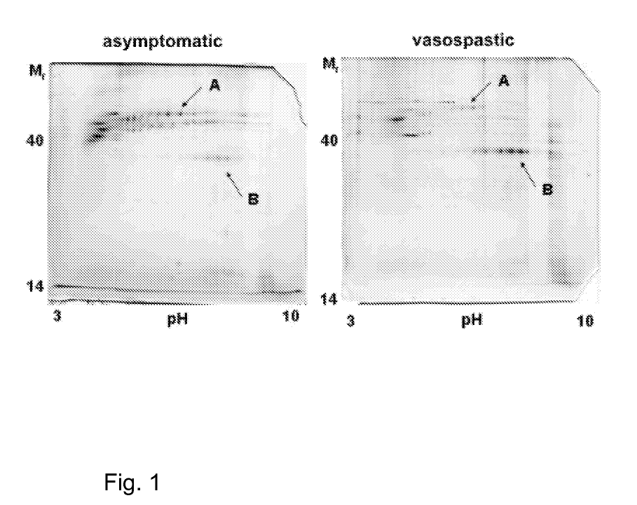

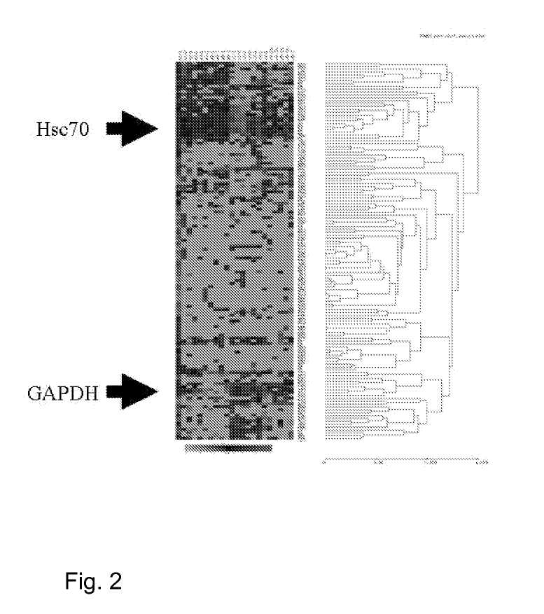

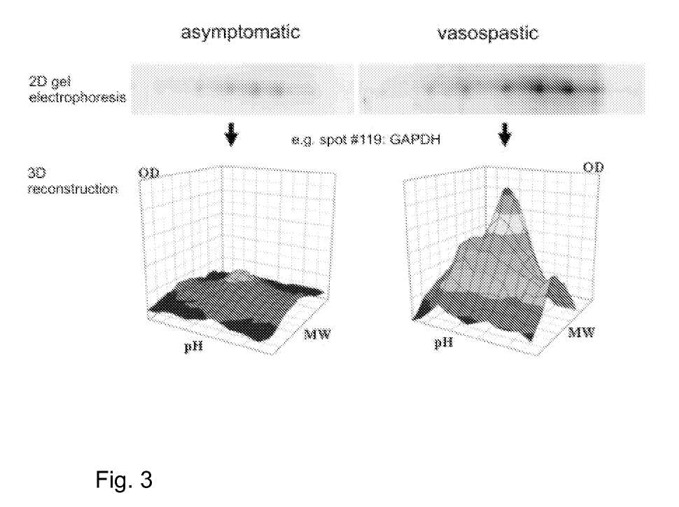

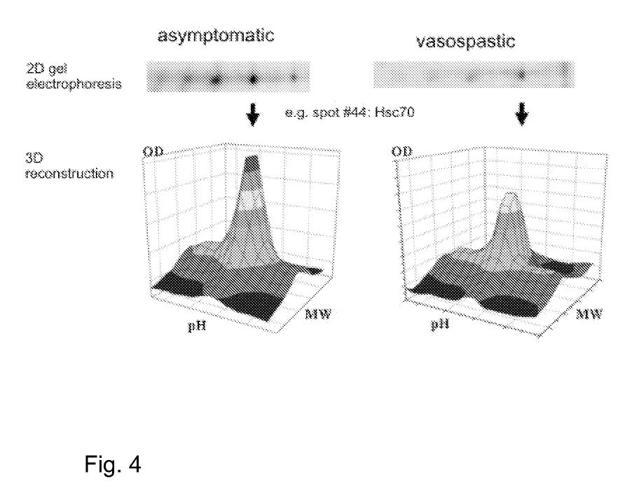

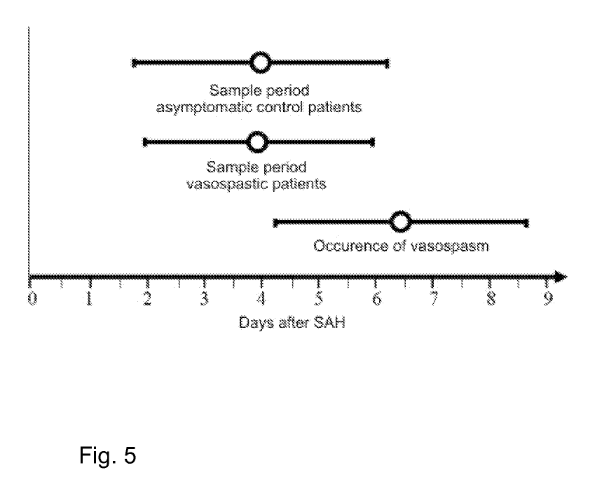

The individual results are shown in the appended FIGS. 1 to 5 and are described in detail in the brief described of the figures.

### LIST OF REFERENCES

1. Benveniste, J Neurochem 52 (1989), 1667-1679
2. Berger et al., Nervenarzt 75 (2004), 113-123
3. Blum et al., Electrophoresis 8 (1987), 93-99
4. Bulliard et al., Biochem J 324 (Pt 2) (1997), 555-563
5. Chuang and Ishitani, Nat Med 2 (1996), 609-610
6. Dastoor and Dreyer, J Cell Sci 113 (Pt 16) (2000), 2845-2854
7. Dietrich and Dacey, Neurosurgery 46 (2000), 517-530
8. Dumont et al., J Neurosurgery 96 (2002), 985-986
9. Dumont et al., Neurosurgery 53 (2003), 123-133
10. Gorg et al., Electrophoresis 21 (2000), 1037-1053
11. Graven et al., J Biol Chem 269 (1994), 24446-24453
12. Graven et al., Am J Physiol 274 (1998), C347-C355
13. Hijdra et al., Stroke 18 (1987), 1061-1067
14. Janjua and Mayer, Curr Opin Crit Care 9 (2003), 113-119
15. Kragten et al., J Biol Chem 273 (1998), 5821-5828
16. Laher and Zhang, J Cereb Blood Flow Metab 21 (2001), 887-906
17. Maurer et al., Proteome Sci 1 (2003), 7
18. Myung et al., Mol Genet Metab 80 (2003), 444-450
19. Nakamura et al., Biochem Biophys Res Commun 290 (2002), 858-864
20. Peerdeman et al., J Neurol 250 (2003), 797-805
21. Persson and Hillered, J Neurosurg 76 (1992), 72-80
22. Rabilloud, Proteomics 2 (2002), 3-10
23. Sakowitz et al., J Cereb Blood Flow Metab 21 (2001), 1067-1076
24. Sarrafzadeh et al., Crit Care Med 30 (2002), 1062-1070
25. Sarrafzadeh et al., Curr Neurol Neurosci Rep 3 (2003), 517-523
26. Sarrafzadeh et al., Stroke 35 (2004), 638-643
27. Saveland et al., Neurosurgery 38 (1996), 12-19
28. Sawa et al., Proc Natl Acad Sci USA 94 (1997), 11669-11674
29. Sirover, J Cell Biochem 66 (1997), 133-140
30. Sirover, Biochim Biophys Acta 1432 (1999), 159-184
31. Sobey, Clin Exp Pharmacol Physiol 28 (2001), 926-929
32. Sunaga et al., Neurosci Lett 200 (1995), 133-136
33. Tatton et al., J Neural Transm Suppl (2000), 77-100
34. Tatton et al., J Neural Transm 110 (2003), 509-515
35. Ungerstedt, Measurement of neurotransmitter release in vivo. 6, Marsden C A ed. Chishester, Wiley (1984), 81-105
36. Ungerstedt, J Int Med 230 (1991), 365-373
37. Unterberg et al., J Neurosurgery 94 (2001), 740-749
38. Vilo J et al., Expression profiler. In: The analysis of gene expression data: Methods and software (2003), Parmigiani G et al., ed. New York, N.Y., Springer Verlag

1-19. (canceled)

20. A method for the in vitro diagnosis of an increased risk of vasospasm in a patient, comprising the steps:
(a) providing a sample from the patient;
(b) determining the quantity of glyceraldehyde-3-phosphate dehydrogenase protein (GAPDH) and/or heat shock cognate 70 kDa protein (Hsc70) in the test sample;
(c) comparing the quantity of GAPDH and/or Hsc70 with a reference value;
wherein a GAPDH quantity that is higher than the reference value and/or an Hsc70 quantity that is lower than the reference value indicates an increased risk of vasospasm.

21. The method of claim 20, wherein the sample is a cerebral microdialysate, cerebrospinal fluid, blood plasma or blood serum.

22. The method of claim 20, wherein the increased risk of vasospasm occurs after a subarachnoid hemorrhage (SAH) or after an intracerebral hemorrhage.

23. The method of claim 20, wherein the determination of the quantity of GAPDH and/or Hsc70 takes place by means of a protein concentration determination.

24. The method of claim 23, wherein the protein concentration determination takes place by means of an antibody or by means of two-dimensional gel electrophoresis.

25. The method of claim 20, wherein the vasospasm is cerebral vasospasm.

26. A test system for detecting an increased risk of vasospasm after a stroke, comprising:
(a) at least one antibody against GAPDH and/or Hsc70;
(b) at least one secondary antibody for detecting antibody/GAPDH and/or antibody/Hsc70 complexes;
(c) optionally means for quantifying the complexes;
wherein a quantity of GAPDH that is higher than the reference value and/or a quantity of Hsc70 that is lower than the reference value indicates an increased risk of vasospasm.

27. A method for identifying a pharmaceutically active substance for the prevention and/or treatment of cerebral vasospasm, comprising the steps:
(a) providing a cell which expresses GAPDH and/or Hsc70;
(b) bringing a test substance into contact with the cell;
(c) measuring the quantity of GAPDH and/or Hsc70;
wherein a reduction in the quantity of GAPDH and/or an increase in the quantity of Hsc70 qualifies the test substance as a pharmaceutically active substance.

28. The method of claim 27, wherein the pharmaceutically active substance is a drug or a medicinal product.

29. A method for screening a substance library to identify a pharmaceutically active substance for the prevention and/or treatment of cerebral vasospasm, comprising the steps:
(a) providing test samples containing cells which express GAPDH and/or Hsc70;
(b) measuring the initial expression quantity of GAPDH and/or Hsc70;
(c) bringing the test samples into contact with candidates from the substance library;
(d) identifying a test sample which expresses less GAPDH and/or more Hsc70 compared to the initial expression quantity;
(e) determining the candidate from the substance library which was brought into contact with the test sample,
wherein the candidate from the substance library is a pharmaceutically active substance.

30. The method of claim 29, wherein the method comprises high-throughput screening (HTS).

31. The method of claim 29, wherein the method comprises calorimetric, luminometric, fluorescence-based or radioactive methods.

32. A pharmaceutically active substance obtainable by the method of claim 29.

33. The pharmaceutically active substance of claim 32, which comprises an inhibitor of GAPDH.

34. The pharmaceutically active substance of claim 32, which comprises an activator of Hsc70.

35. A combination preparation comprising an inhibitor of GAPDH and an activator of Hsc70 for the prevention and/or prophylaxis of cerebral vasospasm.
